(19) United States
(12) Patent Application Publication
(10) Pub. No.: US 2010/0137457 A1
(43) Pub. Date: Jun. 3, 2010

(54) METHOD FOR CONVERSION OF ATMOSPHERIC CARBON DIOXIDE INTO USEFUL MATERIALS

(76) Inventor: Thomas Proger KAPLAN, New York, NY (US)

Correspondence Address: LADAS & PARRY LLP, 26 WEST 61ST STREET, NEW YORK, NY, 10023, US

(21) Appl. No.: 12/494,379

(22) Filed: Jun. 30, 2009

Related U.S. Application Data
(60) Provisional application No. 61/077,321, filed on Jul. 1, 2008.

(51) Int. Cl. C07C 27/06

(52) U.S. Cl. 518/702

(57) ABSTRACT

A method for converting carbon dioxide in a gaseous environment, including air into useful materials by use of renewable energy sources which comprises:
(1) extracting carbon dioxide from a gaseous source using a sorbent such as sodium hydroxide, calcium hydroxide, or potassium hydroxide;
(2) utilizing wind power, solar power, or other renewable energy sources to regenerate said sorbent by membrane cell electrolysis or other similar method, simultaneously producing hydrogen (H₂) gas;
(3) releasing carbon dioxide from said sorbent; and
(4) utilizing the Fischer Tropsch process, Mobil process, ICI process, or related or similar processes to convert carbon oxides to a hydrocarbon concomittantly with or after effecting the reverse water-gas shift reaction to convert said CO₂ and H₂ gas into carbon monoxide for reaction in said Fischer Tropsch process, Mobil process or ICI process.

### FIELD OF THE INVENTION

The present invention relates to a method for converting carbon dioxide existing in a gaseous environment, including air, into useful materials by use of renewable energy sources.

Removal of carbon dioxide from air by use of soda lime, for example in submarines, has been known since the 1940's. Other techniques include absorption in monoethanolamine and use of membranes. However, use of recovered carbon dioxide seems to have been limited. As noted in Quimica Nova vol. 22 n. 2 São Paulo March/April 1999, recovered carbon dioxide has been injected into oil wells to enhance oil recovery. Other uses of carbon dioxide include: refrigerant fluid, cooling agent, food packaging, soldering and moldering agent, fumigant, anti-fire, additive to beverages and water treatment. There have also been proposals for use in synthetic chemistry, for example in production of urea, in the Kolbe-Schmitt reaction to produce 4-hydroxybenzoic acid and as an additive to carbon monoxide in the production of methanol.

U.S. Pat. No. 3,766,027 describes recovery of carbon dioxide from the atmosphere with a chemical sorbent, removal of the CO₂ from the sorbent and regeneration of the sorbent by the application of heat, and conversion of CO₂ into methane by reaction with hydrogen in an electrolysis methanation cell.

U.S. Patent Publication 2008/0115415 (Agrawal and Singh) describes production of synthetic liquid hydrocarbon fuel from carbon containing moieties such as biomass, coal, methane, naphtha as a carbon source and hydrogen from a carbon-free energy source. The biomass can be fed to a gasifier along with hydrogen, oxygen, steam and recycled carbon dioxide. The synthesis gas from the gasifier exhaust is sent to a liquid hydrocarbon conversion reactor to form liquid hydrocarbon molecules. Unreacted CO and H₂ can be recycled to the gasifier along with CO₂ from the liquid hydrocarbon conversion reactor system. Hydrogen can be obtained from electrolysis of water, thermo-chemical cycles or directly by using energy from carbon-free energy sources. Rakesh Agrawal, Navneet R. Singh, Fabio H. Ribeiro, and W. Nicholas Delgass in Proc Natl Acad Sci USA. 2007 Mar. 20; 104(12): 4828-4833 describe a proposal for a hybrid hydrogen-carbon (H₂CAR) process for the production of liquid hydrocarbon fuels wherein biomass is the carbon source and hydrogen is supplied from carbon-free energy. To implement this concept, a process has been designed to co-feed a biomass gasifier with H₂ and CO₂ recycled from the H₂—CO to liquid conversion reactor. In a typical gasifier, oxygen and steam are supplied along with a carbon-containing feed stock. The resulting combustion energy not only provides heat for the endothermic gasification reaction, a majority of which is stored in the CO and H₂ exiting the gasifier, but also compensates for the energy losses from the system. CO₂ is formed in the gasifier from the combustion reaction and through the water-gas shift (WGS) reaction in post-gasifier processing. Whereas in the past it has been common to talk about the possibility of sequestering the resulting CO₂, the H₂CAR process plans to either suppress the formation of this CO₂ or react it with H₂ from a carbon-free energy source such as solar, nuclear, etc. to produce liquid fuel. The reverse WGS reaction of CO₂ with H₂ to form CO and H₂O is an endothermic reaction and requires high temperatures to obtain a reasonable conversion. To simplify the overall process, the authors propose to recycle CO₂ from the H₂—CO to liquid conversion processes (such as a Fischer Tropsch process) to a suitable location in the gasifier. Furthermore, to help drive the thermodynamic equilibrium to the favorable H₂/CO ratio of near two, the proposed process directly feeds H₂ from the carbon-free energy source to the gasifier.

PCT Publication WO 2007/108014 describes a process for producing high octane fuel from carbon dioxide and water. The feedstock for the production line consists of highly concentrated carbon dioxide, extracted as a waste product from industrial processes, and water, which may be of lower quality. Water is electrolyzed into hydrogen and oxygen. The end product can be high octane gasoline, high cetane diesel or other liquid hydrocarbon mixtures suitable for driving conventional combustion engines or hydrocarbon suitable for further industrial processing or commercial use. Products such as dimethyl ether or methanol may also be withdrawn from the production line. The process is emission free and reprocesses all hydrocarbons not suitable for liquid fuel to form high octane products. The heat generated by exotheimic reactions in the process is fully utilized as is the heat produced in the reprocessing of hydrocarbons not suitable for liquid fuel.

Oh-Shim Joo et al in Ind. Eng. Chem. Res., 38 (5), 1808 -1812, 1999 describe use of the reverse-water-gas-shift followed by methanol synthesis reactor to form methanol from CO₂ hydrogenation. Carbon dioxide was converted to CO and water by the reverse-water-gas-shift reaction to remove water before methanol was synthesized. Zinc oxide/alumina catalysts have been developed for this reaction.

### SUMMARY OF THE PRESENT INVENTION

The present invention provides a method by which renewable energy sources are used to convert carbon dioxide existing in a gaseous environment, including air, into useful materials, including fuels such as gasoline, diesel fuel, jet fuel, heating oil, methanol, ethanol, or other organic fuels.

The conversion process is accomplished by (1) extracting carbon dioxide from a gaseous source using a sorbent such as sodium hydroxide, calcium hydroxide, potassium hydroxide, etc.; (2) utilizing wind power, solar power, hydroelectric power, or other renewable energy sources to regenerate said sorbent by membrane cell electrolysis or other similar method, simultaneously producing hydrogen (H₂) gas; and (3) utilizing the Fischer Tropsch process, Mobil process, ICI process, concomittantly with or after effecting the reverse water-gas shift reaction

CO₂+H₂<−−>CO+H₂O

to convert said CO₂ and H₂ gas into useful liquid fuels.

### DETAILED DESCRIPTION OF THE INVENTION

Specifically, the method of the present invention comprises the combination of the steps described below. Each step is based on well established, existing technology. However, the combination of such steps to produce a new environmentally-friendly process for recovery of carbon dioxide and its conversion into useful products is new and provides a means for recycling the carbon dioxide released by burning fuels in, for example, automobiles, trucks, airplanes, and heavy equipment which will release an amount of CO₂ that is equal to the CO₂ that was extracted from the atmosphere by the invention.

The present invention can be used to effectively convert any arbitrary fleet of CO₂-producing engines into a carbon-neutral system powered entirely by renewable energy, without the need to re-engineer the existing fleet. By pairing an arbitrary fleet of CO₂-producing automobiles, trucks, etc. with a fuel production plant of the appropriate capacity which uses the method described, the combined system (consisting of the plant plus the fleet) is rendered carbon neutral: the CO₂ generated by the fleet is completely offset by the CO₂ absorbed in the production plant. The energy used by the fleet is exactly equal to the energy contained in the fuel produced by the plant. Since the fuel produced by the plant is fungible with the fuel used by the fleet, all the energy used by the combined system has effectively been derived from renewable resources. In effect, the invention can be used to convert any existing or future fleet of internal-combustion automobiles, etc. into a carbon neutral system powered entirely by renewable energy—thus obviating the necessity to re-engineer said fleet to achieve reductions in CO₂ emissions. Thus, the invention is a cost-effective substitute for existing research into fuel-cell vehicles, electric cars, etc. In addition, by relying on domestic renewable resources such as wind and solar power, the invention has the potential to replace imported fossil hydrocarbons with domestic, synthetically produced, renewable hydrocarbons, reducing or eliminating the reliance of the US on foreign imported oil.

In one embodiment, the method consists of the following steps, which are each performed simultaneously in a continuous loop cycle.
(1) Extracting CO₂ from ambient air or other gas stream. This can be effected by passively passing such air over a CO₂ sorbent, for example aqueous solutions of alkali metal or alkaline earth metal hydroxides such as sodium hydroxide, potassium hydroxide.

For example, using sodium hydroxide solution as the sorbent, CO₂ is absorbed in a reaction of the form:

CO₂+2NaOH(aq)→Na₂CO₃(aq)+H₂O,

followed by

Na₂CO₃(aq)+CO₂+H₂OΔ2NaHCO₃

The carbon dioxide absorbed in this way may be regenerated periodically on site or transported to a suitable location for regeneration of carbon dioxide from that which has been absorbed at a variety of locations.

Methods for effecting efficient removal of carbon dioxide from the air include those developed by Klaus Lackner and his colleagues, for example those described in PCT publications WO 2006/023743, WO 2006/036396, WO 2006/084008, WO 2006/113674, WO 2007/016271, and W02008/061210 and in U.S. Patent Publications 2006/0186562, and 2006/0289003, the subject matter of all of which are incorporated herein by reference. Such methods may include exposing solvent covered surfaces to air streams where the airflow is kept laminar, or close to the laminar regime. Such methods may involve use of an apparatus, which is a laminar scrubber, comprising solvent covered surfaces situated such that they can be exposed to air streams such that the airflow is dept laminar.
(2) Releasing said CO₂ from the Na₂CO₃ solution by bubbling a gaseous halogen such as chlorine, through the sodium bicarbonate solution produced in step (1), in accordance with a reaction of the form:

Cl₂+2OH⁻→Cl⁻+ClO⁻+H₂O

Na⁺+HCO₃⁻+H⁺+Cl [symbol] CO₂+H₂O+Na⁺+Cl⁻

Or stated alternatively

NaHCO₃(aq)(sodium bicarbonate)+HCl(aq)(hydrochloric acid)→CO₂+NaCl(aq)+H₂O(brine).

One such method is described in PCT Publication WO 2007/018558 (Columbia University), the contents of which are incorporated herein by reference.
(3) Regenerating said sorbent by applying the membrane cell electrolysis process or other comparable process to the brine solution (NaCl(aq)) in accordance with a reaction of the form:

2NaCl+2H₂O→Cl₂+2NaOH+H₂

Said reaction requires the input of electric power in order to proceed. Modem, large scale membrane cell electrolysis plants are widely deployed throughout the world and are used for the commercial production of NaOH and Cl₂. A typical plant producing 100,000 tonnes per annum of NaOH draws about 35 MW of power, which is equivalent to approximately 3,000 kilowatt-hours (kWh) per ton of NaOH produced. If desired and if multiple sorbent locations are used, the regenerated sorbent can then be redistributed to the sorber locations.
(4) Converting CO₂ and H₂ gas into fuels or other useful products utilizing established, existing technologies.

Typically such conversion will be combined with or preceded by at least a partial conversion of carbon dioxide to carbon monoxide with a reaction with hydrogen to produce the desired product. The conversion CO₂ and H₂ gas into liquid fuels utilizes established, existing technologies. As noted above, the reverse-water-gas-shift reaction may be employed to convert carbon dioxide to carbon monoxide which can be used in a variety of reactions to produce hydrocarbons.

Production of long-chain hydrocarbons such as high-cetane diesel, jet fuel, or heating oil may be effected by use of the Fischer Tropsch process by reaction of carbon monoxide and hydrogen in the presence of a Fischer Tropsch catalyst. Conversion of carbon dioxide via the water gas shift reaction into carbon monoxide for use in this reaction is catalyzed by many (but not all) of the same catalysts that promote F-T synthesis, in particular Fe catalyst and other catalysts that contain Fe. The reaction proceeds simultaneously with the main Fisher-Tropsch reaction inside the reactor. In the Fischer-Tropsch reactor, an appropriate amount of steam is introduced so that the stoichiometry results in the production of the appropriate amount of H₂ and the conversion of the appropriate amount of CO₂. Suitable FischerTropsch catalysts are well known and include catalysts based on iron and cobalt, although nickel and ruthenium have also been used. The process was used by Germany in World War II to produce diesel fuel from coal, and has been used in South Africa for the same purposes. Virtually all of South Africa's liquid fuel needs are met by various Fischer Tropsch plants operated by SASOL, the state-run oil company.

As an alternative to proceeding directly from carbon oxides to hydrocarbons, one can also react hydrogen and carbon oxides to produce methanol. The methanol can then be utilized in various industrial processes or for fuel, or it can be further converted to hydrocarbons by known methods. Today, the most widely used catalyst for the production of methanol is a mixture of copper, zinc oxide, and alumina first used by ICI in 1966. At 5-10 MPa (50-100 atm) and 250° C., it can catalyze the production of methanol from carbon monoxide and hydrogen with high selectivity:

CO₂+3H₂→CH₃OH+H₂O

Other techniques for converting carbon dioxide directly to methanol include the electrochemical method of U.S. Pat. No. 4,609,441, enzymatic conversion as described in PCT Publication WO/2007/022504 and catalytic hydrogenation, for example as described by Barrault and Urresta in Comptes Rendus de l'Academie des Sciences Series IIC Chemistry, Volume 2, Number 3, March 1999, pp. 167-174(8).

When gasoline is the desired product, this can be obtained by use of the Mobil process using a ZSM-5 or similar catalyst. Under two standard embodiments of the Mobil process, synthesis gas (CO+H₂) is first converted to either methanol, using techniques described above, or to Fischer-Tropsch products using a Fischer-Tropsch process. By taking advantage of the water gas shift reaction described above

CO₂+H₂ [symbol] CO+H₂O

and adjusting the stoichiometry of the reaction chamber appropriately, either of the above processes can be made to proceed using CO₂ as feedstock in place of CO.

As a second step in the Mobil process, the resulting methanol or Fischer-Tropsch products are reacted over a ZSM-5 catalyst. The reaction products are cooled, distilled, and upgraded, resulting in a high octane gasoline.

Various catalysts for generating ethanol from CO₂/H₂ gas have been proposed; however, none has been deployed on a commercial scale. Methanol may be converted to a hydrocarbon by known methods such as passage over a ZSM-5 catalyst at high temperature, such as 350-450° C.

1. A method for producing hydrocarbons from ambient carbon dioxide which comprises:
(1) extracting carbon dioxide from a gaseous source using a sorbent such as sodium hydroxide, calcium hydroxide, or potassium hydroxide;
(2) utilizing wind power, solar power, or other renewable energy sources to regenerate said sorbent by membrane cell electrolysis or other similar method, simultaneously producing hydrogen (H₂) gas;
(3) releasing carbon dioxide from said sorbent; and
(4) utilizing the Fischer Tropsch process, Mobil process, ICI process, or related or similar processes to convert carbon oxides to a hydrocarbon concomittantly with or after effecting the reverse water-gas shift reaction to convert said CO₂ and H₂ gas into carbon monoxide for reaction in said Fischer Tropsch process, Mobil process or ICI process.

2. The method of claim 1, wherein CO₂ is extracted from ambient air.

3. The method as claimed in claim 2, wherein said extraction is effected by passively passing such air over a CO₂ adsorbent.

4. The method as claimed in claim 3, wherein said absorbent is an aqueous solution of an alkali metal or alkaline earth metal hydroxide.

5. The method as claimed in claim 2, wherein exposing sorbent covered surfaces to air streams where the airflow is kept laminar, or close to the laminar regime

6. The method as claimed in claim 1, wherein CO₂ is released from the absorbent by contact with a halogen.

7. The method as claimed in claim 6, wherein carbon dioxide is captured in said absorbent as a Na₂CO₃ solution and is released by bubbling a gaseous chlorine, through the solution.

8. The method of claim 7, wherein sodium chloride solution formed by reaction of chlorine with the said solution is electrolysed to produce sodium hydroxide.

9. The method of claim 8, wherein sodium hydroxide produced is recycled for adsorption of carbon dioxide.

10. The method of claim 8, wherein said electrolysis is effected by membrane cell electrolysis.

11. The method of claim 1, wherein carbon dioxide released from said sorbent is at least partially converted to carbon monoxide.

12. The method of claim 11, wherein said conversion is effected by the reverse shift water gas reaction.

13. The method of claim 11, wherein carbon oxides are converted to hydrocarbons by reaction with hydrogen in the Fischer Tropsch reaction.

14. The method of claim 11, wherein carbon oxides are converted to methanol.

15. The method of claim 11, wherein carbon oxides are converted to hydrocarbons by reaction with hydrogen to produce methanol which is then converted to hydrocarbon.

16. The method of claim 17, wherein carbon oxides are converted to hydrocarbons by reaction with hydrogen in the ICI reaction.

17. The method of converting ambient carbon dioxide into a hydrocarbon which comprises absorbing carbon dioxide from air into a sodium hydroxide solution to form sodium carbonate, releasing carbon dioxide from said solution by passing chlorine gas through it to produce aqueous sodium chloride, subjecting the carbon dioxide so released to a reverse shift water gas reaction and reaction with hydrogen to produce a hydrocarbon and subjecting said aqueous sodium chloride to electrolysys to produce sodium hydroxide and chlorine and recycling at least some of said sodium hydroxide and chlorine for reuse in the method.
